(19) United States
(12) Patent Application Publication
(10) Pub. No.: US 2024/0423535 A1
(43) Pub. Date: Dec. 26, 2024

(54) BIOMEDICAL DEVICE AND CLINICAL ALGORITHM FOR NICU INFANTS

(71) Applicant: THE REGENTS OF THE UNIVERSITY OF CALIFORNIA, Oakland, CA (US)

(72) Inventors: Caryn BRADLEY, Santa Monica, CA (US); William C TANG, Irvine, CA (US); Joanne LY, Rosemead, CA (US)

(73) Assignee: THE REGENTS OF THE UNIVERSITY OF CALIFORNIA, Oakland, CA (US)

(21) Appl. No.: 18/262,781

(22) PCT Filed: Jan. 25, 2022
(86) PCT No.: PCT/US2022/013699

§ 371 Date: Jul. 25, 2023

Related U.S. Application Data
(60) Provisional application No. 63/141,679, filed on Jan. 26, 2021.

(51) Int. Cl. A61B 5/00; A61B 5/0205

(52) U.S. Cl. CPC A61B 5/4836; A61B 5/0205; A61B 2503/04

(57) ABSTRACT

An apparatus and method for measuring a biosignal of a monitored patient, processing the measured biosignal to generate a treatment protocol for guiding clinical care of the monitored patient, and outputting the treatment protocol for use by a clinical caregiver. In one example, the monitored patient is a preterm infant, the biosignal is a respiratory pattern of the infant during oral feeding, and the treatment protocol provides a recommended treatment regimen setting forth therapeutic interventions and/or medical interventions for a clinical caregiver to follow for achieving a predetermined outcome for improving oral feeding of the monitored infant.

### FIELD OF THE INVENTION

The present invention relates to systems and methods for generating treatment protocols for guiding clinical care of patients, and in particular for guiding the development of oral feeding practices in preterm infants.

### BACKGROUND OF THE INVENTION

An estimated 15 million babies are born prematurely each year. Premature infants have increased risks of health complications and are therefore closely monitored and cared-for in a special ward of the hospital known as the neonatal intensive care unit (NICU). At present, it is expected that 1 in 10 newborns will be born prematurely and admitted to the NICU.

Infants admitted to the NICU must pass a number of tests before being discharged from the ward. Successful oral feeding is the most common barrier to discharge as it involves advanced motor skills, and preterm infants often have difficulty learning to bottle feed due to the dynamic interaction and coordination of a suck-swallow-breathe pattern. Preterm infants often struggle with such motor skills largely due to cardiopulmonary issues, particularly those infants with respiratory distress syndrome. As such, treatment of preterm infants requires consideration of the individual motor skills for sucking, swallowing, and breathing, as well as coordination of those skills during oral feeding.

Achieving safe and efficient oral feeding in preterm infants can be challenging as neurodevelopmental immaturities may contribute to difficulties in achieving cardiorespiratory stability and feeding progress. Modern oral feeding treatments for preterm infants often rely on therapeutic interventions which may include modifications to: a nipple flow rate; an oral feeding schedule; a duration of feeding; a volume of oral intake; a feeding position; and/or feeding pacing. Recourse may also be made to medical interventions, such as respiratory, oxygen, and/or pharmacological support.

The treatments relied on to date present a number of drawbacks. The clinical interventions are conventionally based on behavioral, motoric, and physiologic signs of stress; feeding protocols; and/or feeding outcome measures (e.g., volume intake, duration). However, there is often a lack of data/evidence to support these clinical interventions. The feeding protocols employed may also vary significantly between institutions, and optimal clinical practices are not consistently used. The efficacy of a treatment method is often unclear as outcome metrics are regularly limited to capturing only feeding experience scales and feeding readiness scales, which are subject to individual interpretation and error. Furthermore, the nature of clinical interventions employed often vary substantially between institutions and individual practitioners.

Accordingly, there remains a need for clinical systems and methods that provide reliable and efficient treatments for achieving oral feeding of preterm infants.

### SUMMARY OF THE INVENTION

The present invention is inclusive of a system and method for guiding clinical treatments, the system comprising input circuitry for receiving one or more biosignals from at least one biosignal sensor that measures biosignals of a monitored patient; at least one processor adapted for processing received biosignals to generate a treatment protocol for the monitored patient; and output circuitry for outputting a treatment protocol.

The input circuitry is adapted to receive a biosignal as a waveform, and the processor is adapted to process the biosignal waveform to clean useful data from the biosignal waveform, calculate a health score (sample entropy score) from the cleaned biosignal waveform, and display clinical support to clinicians, which may include generation of a treatment protocol, based on the health score via a clinical algorithm that applies predefined rules for determining a recommended regimen for guiding clinical care of the monitored patient.

In one example, the input circuitry is adapted to receive a biosignal waveform in the form of a respiratory waveform, as measured from a preterm infant during oral feeding activity; and the processor is adapted to generate a treatment protocol that sets forth recommended therapeutic interventions and/or medical interventions for a clinical caregiver to follow for achieving a predetermined outcome for improved oral feeding of the monitored infant.

The system may include one or more biosignal sensors connected to the input circuitry and adapted for non-invasive monitoring of the infant, non-limiting examples of the one or more biosignal sensors being selected from: a body position sensor; a pulse oximeter; a blood pressure sensor; a temperature sensor; an electromyography (EMG) sensor; an electrocardiogram (ECG) sensor; an airflow sensor; and a galvanic skin response (GSR) sensor.

Both the foregoing general description and the following detailed description are exemplary and explanatory only and are intended to provide further explanation of the invention as claimed. The accompanying drawings are included to provide a further understanding of the invention; are incorporated in and constitute part of this specification; illustrate embodiments of the invention; and, together with the description, serve to explain the principles of the invention.

### BRIEF DESCRIPTION OF THE DRAWINGS

Further features and advantages of the invention can be ascertained from the following detailed description that is provided in connection with the drawings described below:

FIG. 1 shows a computing system for use with systems of the present invention;

FIG. 2 shows a method of practicing the present invention with a system of FIG. 1;

FIG. 3 shows impedance measurements, as a biosignal, for informing breathing of an infant;

FIG. 4 shows one example of a waveform that may be generated by the system of FIG. 1 in accord with the method of FIG. 2;

FIG. 5 shows steps for cleaning the waveform of FIG. 4 in accord with the method of FIG. 2;

FIG. 6 shows a machine learning workflow for development of a clinical algorithm;

FIG. 7 shows an example of the system according to the present invention, with a system-on-chip construction;

FIG. 8 shows an example of the system according to the present invention for generating histogram reports, with a system-on-chip construction; and

FIG. 9 shows an example of an oxygen saturation histogram generated in accord with the present invention.

### DETAILED DESCRIPTION OF THE INVENTION

The following disclosure discusses the present invention with reference to the examples shown in the accompanying drawings, though does not limit the invention to those examples.

The use of any and all examples, or exemplary language (e.g., “such as”) provided herein is intended merely to better illuminate the invention and does not pose a limitation on the scope of the invention unless otherwise claimed. No language in the specification should be construed as indicating any non-claimed element as essential or otherwise critical to the practice of the invention, unless made clear in context.

As used herein, the singular forms “a,” “an,” and “the” include plural referents unless the context clearly dictates otherwise. Unless indicated otherwise by context, the term “or” is to be understood as an inclusive “or.” Terms such as “first”, “second”, “third”, etc. when used to describe multiple devices or elements, are so used only to convey the relative actions, positioning and/or functions of the separate devices, and do not necessitate either a specific order for such devices or elements, or any specific quantity or ranking of such devices or elements.

The word “substantially”, as used herein with respect to any property or circumstance, refers to a degree of deviation that is sufficiently small so as to not appreciably detract from the identified property or circumstance. The exact degree of deviation in a given circumstance will depend on the specific context, as would be understood by one having ordinary skill in the art.

Use of the terms “about” or “approximately” are intended to describe values above and/or below a stated value or range, as would be understood by one having ordinary skill in the art in the respective context. In some instances, this may encompass values in a range of approx. +/−10%; in other instances, there may be encompassed values in a range of approx. +/−5%; in yet other instances values in a range of approx. +/−2% may be encompassed; and in yet further instances, this may encompass values in a range of approx. +/−1%.

It will be understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof, unless indicated herein or otherwise clearly contradicted by context.

The terms “individual”, “host”, “subject”, and “patient”, as may be used interchangeably herein, refer to a mammal, including, but not limited to, primates, for example, human beings, as well as rodents, such as mice and rats, and other laboratory animals.

Recitations of a value range herein, unless indicated otherwise, serves as a shorthand for referring individually to each separate value falling within the stated range, including the endpoints of the range, each separate value within the range, and all intermediate ranges subsumed by the overall range, with each incorporated into the specification as if individually recited herein.

Unless indicated otherwise, or clearly contradicted by context, methods described herein can be performed with the individual steps executed in any suitable order, including: the precise order disclosed, without any intermediate steps or with one or more further steps interposed between the disclosed steps; with the disclosed steps performed in an order other than the exact order disclosed; with one or more steps performed simultaneously; and with one or more disclosed steps omitted.

The present invention is inclusive of systems and methods for monitoring and measuring bioelectric signals of a subject, and using the measured results to generate a treatment protocol providing a recommended regimen for guiding clinical care of the subject. The treatment protocol may include one or more clinical interventions (e.g., therapeutic and/or medical interventions) for achieving a desired clinical outcome.

The present invention adopts non-linear methods and adaptive subroutines trained from large amounts of data to perform pattern identification, feature extraction, and variability assessment. This data is then used to generate a set of mathematical “health scores” classifying an adaptability and/or rigidity of a patient's condition (e.g., an infant's breathing pattern). The health scores, along with other physiologic parameters and clinical variables, are then used to generate the treatment protocol with recommendations having statistical significance for bringing patient treatment to a clinical decision endpoint.

In one example, the present invention may be employed to monitor breathing and feeding patterns of a preterm infant, and for generating a treatment protocol in the form of a feeding regiment for guiding oral-feeding practices based on respiratory waveforms and other associated physiologic metrics, with a goal of improving oral-feeding performance as well as an overall clinical outcome.

The following disclosure addresses examples in which systems and methods according to the present invention are employed to monitor and measure the breathing pattern of a preterm infant, and to generate a treatment protocol for guiding oral feeding of the infant. In such examples, a biomedical system collects, downloads, cleans, analyzes, and interprets respiratory data and uses the same, along with other physiologic parameters and feeding outcome measurements, to create a treatment protocol for neonatal therapists and neonatologists to guide therapeutic and medical interventions for achieving an oral feeding target with the infant that will satisfy prerequisites for discharge from an NICU. However, it will be understood that this is but one non-limiting example of the inventions.

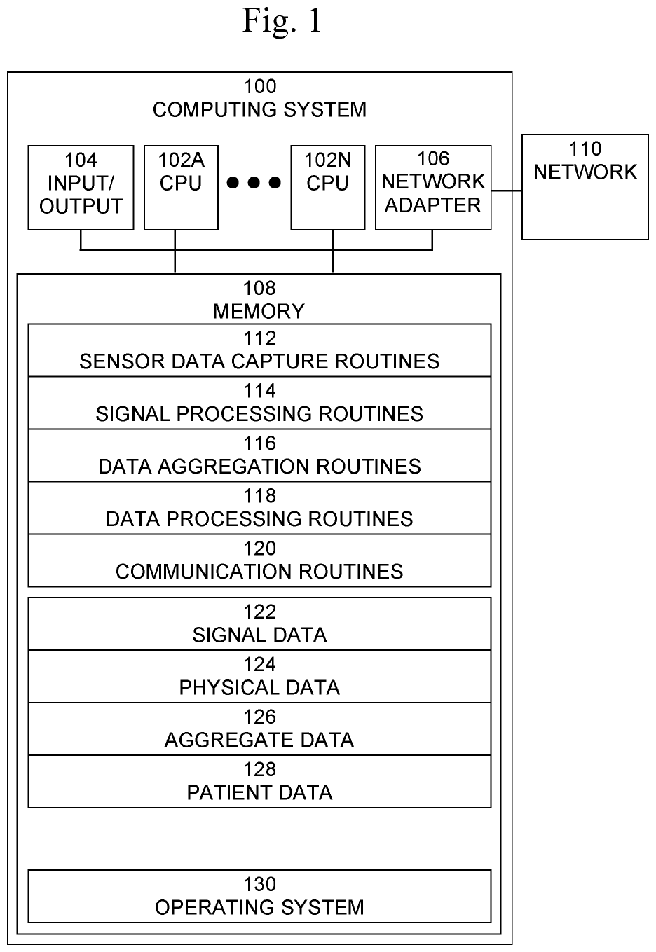

An exemplary block diagram of a computer-implemented system 100, in which processes involved in the embodiments described herein may be implemented, is shown in FIG. 1. Generally, the system 100 includes input circuitry 104 for receiving a waveform from at least one biosignal sensor; a memory 108 for storing data and operational programs; at least one processor 102 for processing stored data and executing stored programs to generate a treatment protocol; and output circuitry 104 for outputting the treatment protocol. In the illustrated example, input and output circuitry are provided as a dual purpose input/output circuitry 104, though it will be understood that the input and output circuitries may instead be provided separately and independent from one another. Likewise, while the illustrated example shows the processor 102 as comprising a plurality of processors 102A-102N, it will be understood that there may instead be a single processor 102.

System 100 is typically a programmed general-purpose computer system, such as an embedded processor, system on a chip, personal computer, workstation, server system, and minicomputer or mainframe computer. System 100 may include one or more processors (CPUs) 102A-102N, input/output circuitry 104, a network adapter 106, and a memory 108. CPUs 102A-102N execute program instructions to carry out functions of the present invention. Typically, CPUs 102A-102N are one or more microprocessors, microcontrollers, processor in a System-on-Chip, etc. FIG. 1 illustrates an embodiment in which system 100 is implemented as a single multi-processor computer system, in which multiple processors 102A-102N share system resources, such as memory 108, input/output circuitry 104, and network adapter 106. However, in other examples the system may be implemented as a plurality of networked computer systems, which may be single-processor computer systems, multi-processor computer systems, or a mix thereof.

Input/output circuitry 104 provides the capability to input data to and, and output data from, system 100. The input/output circuitry 104 includes connections for interfacing with an array of medical diagnostic and treatment tools, which may include, though are not limited to: biosignal sensors such as a body position sensor; a pulse oximeter; a blood pressure sensor; a temperature sensor; an electromyography (EMG) sensor; an electrocardiogram (ECG) sensor; an airflow sensor; a galvanic skin response (GSR) sensor; and one or more electrodes. The input/output circuitry 104 may also include connections for non-medical input devices, such as sensors, microphones, keyboards, mice, touchpads, trackballs, scanners, etc.; and non-medical output devices, such as speakers, video adapters, monitors, printers, etc.; as well as dual-purpose input/output devices, such as, modems, etc. The input/output circuitry 104 may communicate with the network adapter 106 to interface the system 100 with a network 110, which may include any public or proprietary LAN or WAN, Bluetooth wireless communication module, including, but not limited to the Internet.

The memory 108 stores program instructions that are executed by, and data that are used and processed by, CPU 102A-102N to perform the functions of system 100. Memory 108 may include, for example, electronic memory devices, such as random-access memory (RAM), read-only memory (ROM), programmable read-only memory (PROM), electrically erasable programmable read-only memory (EEPROM), flash memory, etc., and electro-mechanical memory, such as magnetic disk drives, tape drives, optical disk drives, etc., which may use an integrated drive electronics (IDE) interface, or a variation or enhancement thereof, such as enhanced IDE (EIDE) or ultra-direct memory access (UDMA), or a small computer system interface (SCSI) based interface, or a variation or enhancement thereof, such as fast-SCSI, wide-SCSI, fast and wide-SCSI, etc., or Serial Advanced Technology Attachment (SATA), different versions of Universal Serial Bus (USB), or a variation or enhancement thereof, or a fiber channel-arbitrated loop (FC-AL) interface.

The contents of memory 108 may vary depending upon the function that system 100 is programmed to perform. One of skill in the art will recognize that routines, along with the memory contents related to those routines, may not typically be included on one system or device, but rather are typically distributed among a plurality of systems or devices, based on well-known engineering considerations. Examples of the present invention may employ any and all such arrangements.

As shown in the example of FIG. 1, memory 108 may include sensor data capture routines 112, signal processing routines 114, data aggregation routines 116, data processing routines 118, communication routines 120, signal data 122, physical data 124, aggregate data 126, patient data 128, and operating system 130. For example, sensor data capture routines 112 may include routines to receive and process signals from one or more sensors of various types, such as those described above, to form signal data 122. Signal processing routines 114 may include routines to process signal data 122 to form physical data 124. Data aggregation routines 116 may include routines to process physical data 124 to generate aggregate data 126. Data processing routines 118 may include routines to process physical data 124, aggregate data 126, and/or patient data 128. Communication routines 120 may include routines for controlling communications between the system 100 and other devices, system, and networks. Operating system 130 provides overall system functionality.

Examples of the present invention may use systems 100 that provide multi-processor, multi-tasking, multi-process, and/or multi-thread computing, though other examples may use systems 100 that provide only single processor, single thread computing. Multi-processor computing involves performing computing using more than one processor.

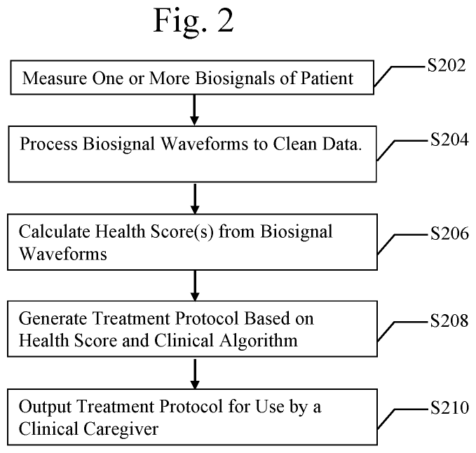

An example of a method according to the present invention, as shown in FIG. 2, includes measuring at least one biosignal of a patient (step S202); processing biosignal waveforms to clean useful data of noise, artifacts, and/or anomalies (step S204); calculating a numerical value indicative of the at least one biosignal waveform (step S206); generate a generate a treatment protocol based on the health score and a clinical algorithm (step S208); and outputting the treatment protocol for use by a clinical caregiver (step S210).

In operation, the system 100 monitors and measures at least one biosignal of a patient (step S202). Measured biosignals may include, though are not limited to, signals that inform: respiratory rates (RR) and patterns; heart rates (HR) and patterns; brainwave patterns; muscle contraction patterns; eye movement patterns; partial pressure of oxygen (PaO₂), peripheral capillary oxygen saturation (SpO₂) patterns, arterial oxygen saturation (SaO₂) patterns, percent hemoglobin oxygen saturation patterns, oxygen saturation histogram patterns, blood pressure patterns, body temperature patterns, transcutaneous carbon dioxide tension (tcPCO₂), oxygen tensions (tcPO₂), and electrodermal activity patterns.

A system 100 according to the present invention may interface with currently available medical diagnostic and treatment tools and equipment to monitor and measure biosignals at a near continuous rate (e.g., every 16 ms), with the measured biosignals recorded as waveforms. In one example, the system 100 may operate as an auxiliary unit that collects respiratory data for an infant through a bedside cardiopulmonary monitoring system and/or pulse oximetry monitoring system that provides non-invasive monitoring of the patient (e.g., Philips Intellivuc) to measure and record one of more biosignals, and may communicate with a data collection system for the export of data (e.g., ixTrend, available from ixitos GmbH, Berlin, Germany; MATLAB®, available from MathWorks, Inc., Natick, MA; Vital Recorder, Sci Rep. 2018 Jan. 24; 8 (1): 1527). The system 100 may also perform signal pre-processing and sample entropy calculations once recording is completed.

In some examples, a system 100 according to the present invention may operate as an independent, stand-alone unit that may optionally be made portable. In such examples, the system 100 may measure biosignals directly from a preterm infant, and may process and analyze a health score, all without support from other existing monitors and systems. Such a system 100 may also send a health score to a designated virtual machine that utilizes an AI-based interpretative model to determine rigidity of the preterm infant's oral feeding performance, with results displayed on an auxiliary screen (e.g., a touchscreen) of the system 100. Alternatively, rather than communicating with a separate virtual machine, the system 100 may itself use an AI-based predictive model to identify and extract key clinical variables from an electronic health record, and employ a clinical algorithm to generate clinical oral feeding assessments that are recorded directly into the system with results again displayed on an auxiliary screen of the system 100. In yet further examples, the system 100 may also serve as a diagnostic support device that generates and establishes therapeutic recommendations that are also displayed on an auxiliary screen of the system 100

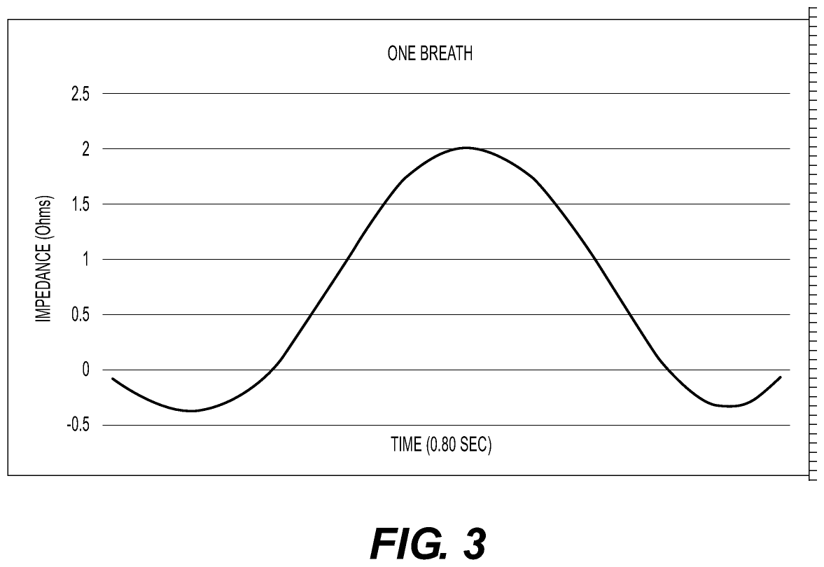

In examples where biosignals are collected based on respiratory data, a respiratory waveform may be collected as a measurement of transthoracic electrical impedance and/or an indirect measurement of respiratory variability. Transthoracic electrical impedance may be achieved by measuring resistance via signals obtained from two electrocardiogram electrodes placed on an infant's chest, with the electrodes measuring electric potential generated by electrical activity in cardiac tissue. These values may then be used to calculate a relative change in impedance based on chest movement that occurs during inspiration and expiration. Generally, there will be detected changes in impedance based on changes in an air-to-tissue ratio when the infant inhales and exhales, with a relative increase in impedance when air enters the lungs during an inspiratory effort and a relative decrease in impedance when air exits the lungs during an expiratory effort. An example of impedance measurements as informing breathing of an infant is shown in FIG. 3, which illustrates impedance measurements for a single breath.

A further discussion of respiratory waveforms is provided by Bradley, C., “Nonlinear Methods of Analysis to Examine Respiratory Waveform Variability During Oral Feeding in Preterm Infants”, Int. J. Medical Engineering and Informatics, 2017, Vol. 9, No. 3, pages 284-298. Reference is also made to Lec, H., et al., “A New Algorithm for Detecting Central Apnea in Neonates”, Physiol. Meas. 2012, Vol. 33, pages 1-17; and Mohr, M. A., et al., “Quantification of Periodic Breathing in Premature Infants, Physiol. Meas., 2015, Vol 36, pages 1415-1427, each of which are incorporated by reference herein in their entirety.

In examples where the monitored subject is a preterm infant, the bedside monitoring system may measure respiratory waveform variability (breathing patterns), which may then be processed to provide a non-linear analytic analysis (a statistical measure of the change in complexity of physiologic processes) for use in clinical care of the infant within the NICU. Respiratory waveform variability plots transthoracic impedance over time and is characterized by either adaptability or rigidity of the breathing pattern. Such a non-invasive, bedside data collection means will enable real-time monitoring of the infant during clinical treatments, such as bottle feeding and breast feeding, as well as during medical treatments such as changes in respiratory support devices.

Measured biosignals are recorded as waveforms, and may be recorded individually or as a multiplexed signal with subsequent signal processing (e.g., independent component analysis; fast Fourier transform; etc.), as well as data processing to clean useful data from background noise, artifacts, and anomalies (step S204). For example, when monitoring a respiratory pattern of a preterm infant, the respiratory waveform may include movement artifacts based on the infant's movement during feeding, as well as cardiac overlay artifacts based on heartbeat signals. Preferably, such artifacts are removed so as to better observe the respiratory waveform data.

The system 100 may collect biosignal data (e.g., ECG, SpO₂, etc.) from an external system, such as a Philips Multi-Measurement Server, and may store those biosignals for data processing. Alternatively, the system 100 may collect biosignals directly from a preterm infant via a number of electrode inputs. Biosignal data may also be supplemented by other clinical data (e.g., oral assessments of a patient) as obtained by a user and entered into the system through a user interface.

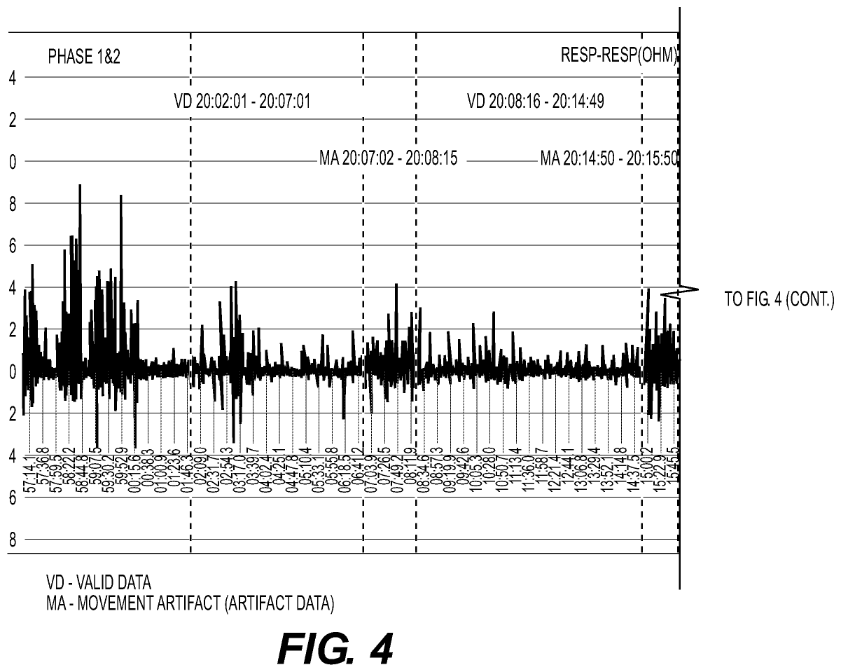

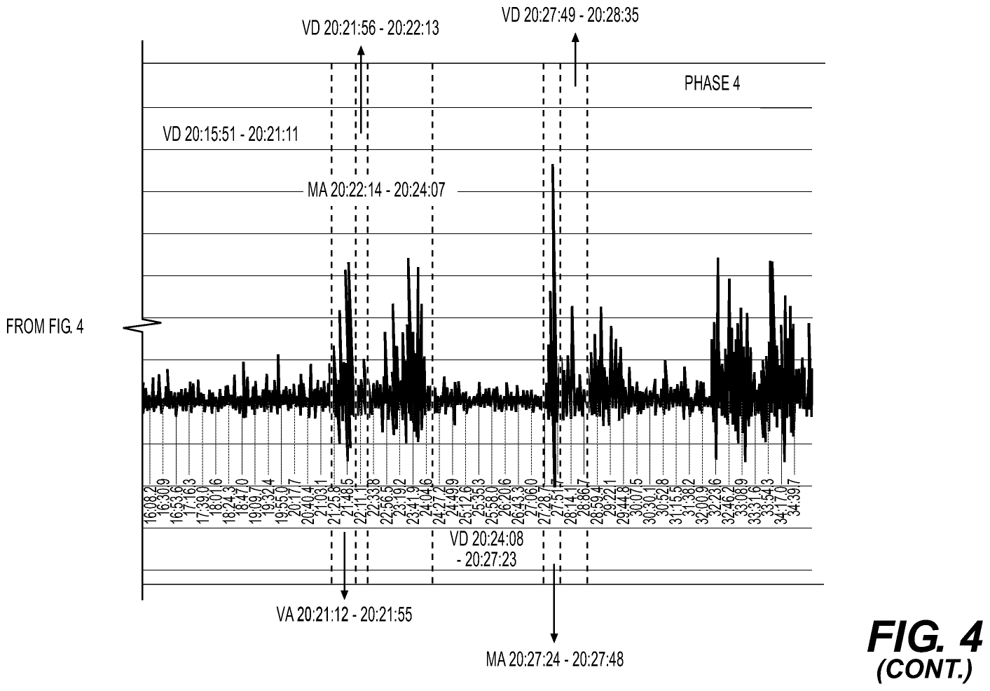

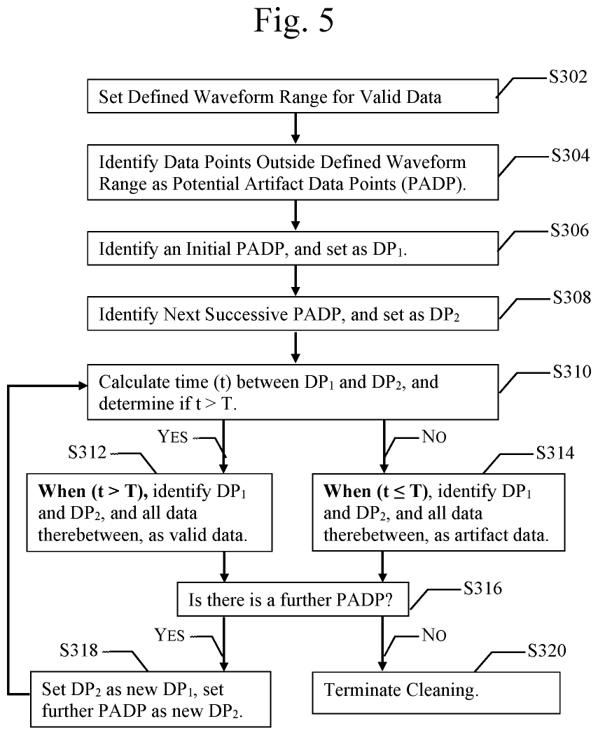

Biosignals received at the system 100 are extracted from the memory, interpreted, and cleaned using Python-based digital filters to remove noise (e.g., removal of cardiac and motion artifacts from respiratory waveform variations). FIG. 4 shows one example of a respiratory waveform that is cleaned to separate movement artifacts from valid data, and FIG. 5 presents a data clipping method for cleaning the waveform to remove artifact data. In a step S302, a waveform range is defined for valid data based on a Hilbert transform of instantaneous frequency, in that values within the set range are deemed valid data and values outside the set range are deemed potential artifact data. In the example shown in FIG. 4, the defined waveform range is set to (−1≤y≤3) as values within this range are considered indicative of respiratory-related movements, whereas values outside this range are considered potential non-respiratory movements (e.g., artifact data), such as movement of the infant's limbs, positon change of the infant, burping of the infant, pauses in feeding, or movement of the infant while held by a caregiver.

Following setting of the defined waveform range, an initial assessment is made to identify all data points in the waveform that are outside the defined waveform range (step S304), with all such data points deemed potential artifact data points (PADP). Once the potential artifact data points are identified, an initial PADP is then set as a first data point DP₁ (step S306), the next successive PADP thereafter is set as a second data point DP₂ (step S308), and a time (t) between the two data points DP₁ and DP₂ is then calculated (step S310).

If the time (t) between the data points DP₁ and DP₂ is greater than a predetermined threshold time (e.g., T>1.5 minute), then the data points DP₁ and DP₂, and all data therebetween, is deemed valid data (step S312). Alternatively, when the time (t) between two successive data points DP₁ and DP₂ is less than or equal to the predetermined threshold time (e.g., T≤1.5 minutes), then both data points DP₁ and DP₂, and all data therebetween, is deemed artifact data. (step S314).

Following the determination of valid or artifact data based on a time duration (as in steps S310-S314), it is then determined if there are any further data points that were deemed potential artifact data points (PADP) in step S304. If there is not any further PADP, then data cleaning terminates (step S320). If there is a further PADP, then the previously identified data point that was previously set as the second data point DP₂ is updated to be the new first data point DP₁ and the next successive PADP is set as a new second data point DP₂ (step S318), and data cleaning continues by repeating the time calculation between the newly set data points DP₁ and DP₂ (step S310), as well as each successive step thereafter (S312-S318), until the process terminates upon a determination that there are not any further PADP (step S320), at which time the entirety of the waveform will thus be cleaned of artifact data.

In the foregoing example, the defined waveform range ([−1, 3]) is set based on the expected values for a respiratory movement and the predetermined threshold time (T of 1.5 minutes) is chosen based on expected durations of an infant's non-respiratory movements. The defined waveform range is set to an appropriate value range that is predetermined to distinguish between valid and artifact data, and that the threshold time is likewise set to an appropriate value that is predetermined to distinguish between the intended data for measurement and unintended artifact data, based on the particular monitoring being performed with a given patient. It will be understood that the defined waveform range and threshold time are specific to the time the data is collected from a specific patient, and that these values are expected to differ from patient to patient, depending on the gestation age, degree of oral feeding readiness, and other factors; and that this data may also change for a single patient depending on the maturation progress of the patient at the time of data collection. Thus, it is recommended the defined waveform range and threshold time be determined for each specific dataset. It will also be understood that though the foregoing example discusses a cleaning method for distinguishing between valid respiratory movement data and non-respiratory artifact movement data, a similar cleaning process may be used for distinguishing between valid and artifact data for biosignal data types other than respiratory movement data (e.g., heart rate data).

Following data cleaning, at least one numerical value is then calculated based on the cleaned biosignal waveform through a Python code (step S206). This calculated numerical value may be referred to herein as a “health score” or a “sample entropy score”, and may be represented in any chosen range of values (e.g., 0≤ score≤1). Systems according to the present invention may employ non-linear methods and adaptive subroutines that are trained from large training data sets for performing pattern identification, feature extraction, and variability detection to generate a set of health scores that classify and categorize the adaptability or rigidity of one or more biosignals. A health score is a measurement of the complexity of a physiological time-series of a biosignal, with reduced health scores representing greater rigidity and lesser adaptability, and higher health scores representing greater adaptability and lesser rigidity.

A health score (sample entropy score, “SampEn”) may be calculated with the following equation:

$$SampleEn = {Φ}^{m} ( r ) = \frac{1}{N - m + 1} {\sum }_{i = 1}^{N - m + 1} log ( {C}_{i}^{m} ( r ) ) ,$$

in which N represents a length of a total time series of a dataset, m represents the length of the data segment being compared (i.e., the epoch length), r represents a pre-established criterion for identifying similarity between separate data points, and Cᵢ^{m} represents a conditional probability.

Parameters for the calculation of a health score may be defined based on the biological signals that are being captured. For example, in instances where a preterm infant is monitored for the capture of respiratory waveforms via transthoracic impedance signals during oral feeding, a health score may be calculated with parameters of m=5 and r=0.2 breaths/min, with N typically ranging from 17,000 to 56,000 data points per feeding. In such an example, the value of m=5 may be chosen, for example, based on characteristics of respiratory waveform patterns, with the tolerance r calculated as a factor of the standard deviation (σ), with r=0.20, with a standardized deviation of σ=1 yielding a value of r=0.2.

The one or more health scores, along with other physiologic parameters and clinical variables, is then input to a clinical algorithm that applies a series of pre-defined rules to create a treatment protocol that provides a recommended regimen for guiding clinical care of the monitored patient (step S208). The clinical algorithm is a predictive tool for providing guidance on clinical treatments, and may be a product of a cloud-based data collection system, and may be self-learning and adaptive to improve predictive accuracies. Preferably, the clinical algorithm provides information relative to other clinical applications (e.g., pediatric/neonatal clinical diagnosis) for addressing a variety of clinical needs.

The additional physiologic parameters may include, though are not limited to, metrics such as heart rate, respiratory rate, oxygen saturation, etc. Clinical variables may vary depending on the clinical treatment to be applied. In the example of treating a preterm infant, non-limiting examples of clinical variables may include feeding outcome data such as volume intake, duration of feeding, etc. The clinical algorithm may apply variable weights to the one or more physiologic parameters and/or clinical variables that reflect the varying impact that each may have on the patient and/or the health score of the patient. The treatment protocol generated is based on the health scores reflecting the current patient status, as well as the additional parameters and variables, and will preferably provide recommendations as to therapeutic and/or medical interventions customized to the patient, as well as a predicted treatment outcome.

In some examples, the system 100 may have a user interface adapted for user input of data and commands, and the clinical algorithm (step S208) may include rules that require a user to input additional data for use in generating a treatment protocol. For example, the clinical algorithm may require a user to input other clinical variables independent from the measured biosignal, which may include patient-specific data (e.g., age, weight, medical history, etc.) and/or clinic-specific data (e.g., medications, tools, and specialists available at treatment site). In the example of a preterm infant, patient-specific data may include, though is not limited to: antenatal/perinatal respiratory history; social determinants of health; infant demographics; maternal data; neonatal disease severity score; and measures of feeding performance.

The resulting treatment protocol is output from the system 100 via an output device connected to the output circuitry 104 for use by a clinical caregiver as a recommended treatment regimen for the monitored patient (step S210). Preferably the system 100 includes a graphical user interface for visualization of biosignal measurements, the recommended treatment regimen, and predicted treatment outcome.

As one non-limiting example, the clinical algorithm may be adapted for monitoring a preterm infant for improving oral-feeding readiness. In such an example, the clinical algorithm will use respiratory waveforms, and variabilities identified therein, to quantify a relationship between the infant's swallowing and breathing patterns that informs the infant's performance in coordinating breathing with feeding. By assessing coordination between breathing and feeding, the system is capable of generating standardized treatment protocols that increase uniformity in clinical practices for developing personalized feeding practices from one infant to another.

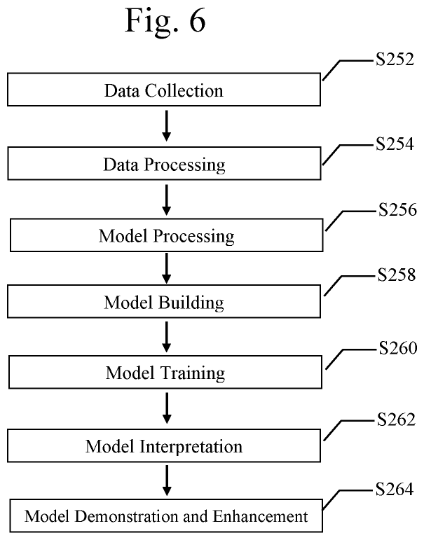

FIG. 6 shows one example of a machine learning workflow for development of a clinical algorithm in accord with the present invention. In this example, a clinical algorithm is developed through a number of sequential steps inclusive of: data collection S252, data processing S254, model processing S256, model building S258, model training S260, model interpretation S262, and model demonstration and enhancement S264.

In a data collection step S252, a data collection program extracts patient data from maternal and infant medical records, such as an Electronic Health Record (EHR), and sends the extracted data to an AI-based computational algorithm. The computational algorithm compiles and combines each infant's relevant EHR data with their respective sample entropy measurements, corresponding to each respiratory waveform measured for the respective infant, as well as the feeding outcome for that respective infant, and creates a feature matrix.

The feature matrix is inclusive of a number of relevant factors, such as infant demographics, maternal factors, physiological factors, and measures of feeding performance. Infant demographics may include, though are not limited to: gestational age, postmenstrual age, birth weight, birth length, birth history, congenital anomalies, genetics (e.g., infant of a diabetic mother), and infant clinical history (e.g. respiratory, cardiovascular, gastrointestinal, hematologic, neurologic activity). Maternal factors may include, though are not limited to: maternal age, gravidity, parity, pre-eclampsia, obesity, and asthma. Physiology factors may include, though are not limited to: heart rate, respiratory rate, oxygen saturation histograms (SpPO₂, SaO₂), and a sample entropy score. Measures of feeding performance may include, though are not limited to: proficiency, volume intake, duration, rate of milk transfer, and one or more feeding readiness scores (e.g. behavioral state regulation, feeding quality, caregiver interventions, etc.).

In a data processing step S254, a data cleaning program cleans and formats the extracted data in preparation for analysis. The computational algorithm identifies any discontinuities or irregularities in the dataset and formats variables to re-level or cut categorical features for any sparsely represented categories as necessary for transforming the corresponding feeding outcomes into a binary feeding outcome variable indicating “success” vs “non-success”.

In a model processing step S256, a data processing program checks the feature matrix for instances of multicollinearity among independent variables and modifies the dataset as needed to remove instances of high multicollinearity, as well as any low variance variables contributing thereto. The algorithm may implement techniques such as variable centering or principal component analysis for this purpose. The algorithm may also employ preliminary logistic regression models to analyze the results and determine the need for multicollinearity invariant regression models, such as ridge or lasso, to identify any relevant interactions that should be included in the model.

In a model building step S258, the computational algorithm builds an Akaike information criterion (AIC)-stepwise variable selection logistic regression model with the binary feeding outcome variable as the dependent variable, extracting the model coefficients and each of their corresponding p-values and standard errors to identify and select the best features.

In a model training step S260, the computational algorithm uses a data training set to trend features (e.g., data learning and fine tune parameters) and assess the model's predictive power. This may be done, for example, by executing the model, evaluating with a leave-one out cross-validation scheme, and checking the area-under-the-curve, as well as plot a receiver operating characteristic (ROC) curve.

In a model interpretation step S262, the computational algorithm uses machine learning to determine significant variables (e.g., meaningfully large effect sizes, etc.) and check model goodness of fit metrics, such as the coefficient of determination (R²) and determine whether the model requires further alternations and re-formatting. This validation of the performance model may be repeated as necessary until a consistent predictive model is achieved.

In a model demonstration and enhancement step S264, the feature matrix is fed through the computational algorithm for interpretation and validation. The resulting computational algorithm is a self-learning and adaptive algorithm that improves predictive accuracies while enabling generation of generally standardized treatment protocols that may be adapted for individualized clinical care of specific patients.

While the foregoing example addresses the development of a clinical algorithm for use in promoting feeding outcome in preterm infants, it will be understood that it may likewise be adapted for development of a clinical algorithm for use in promoting improvements in other pediatric, neonatal and general clinical diagnostic practices.

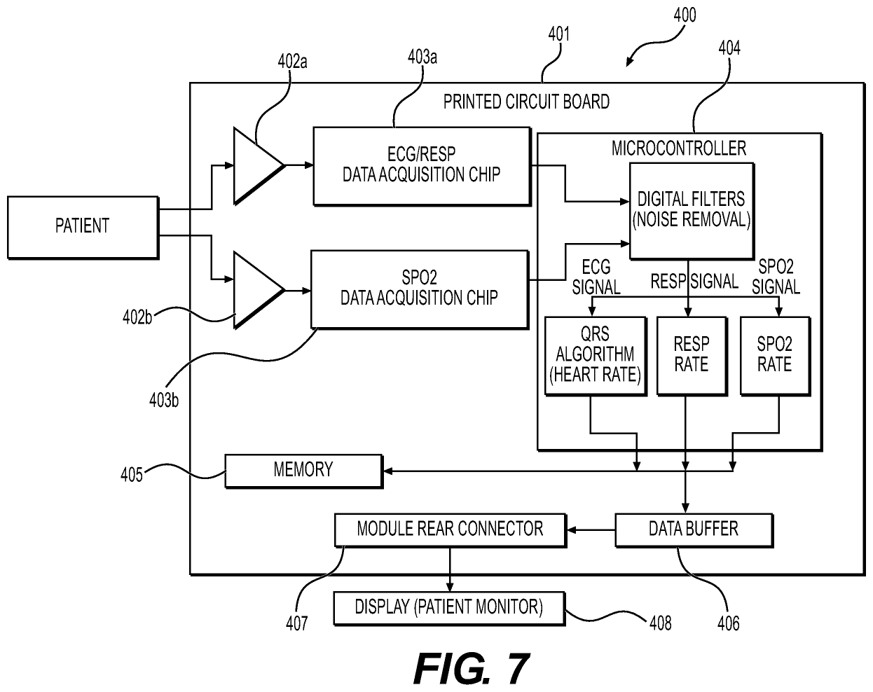

FIG. 7 shows an example of a system 400 according to the present invention, in the form of a system-on-chip built on a printed circuit board 401. In this example, a patient, such as a preterm infant, is monitored for capture of ECG/respiratory signals through a number of electrodes applied to the infant's chest and abdomen, and the capture of an oxygen saturation (SpO₂, SaO₂) signal through an electrode cuff attached to the infant's foot. The ECG/respiratory signals are amplified by a first low-noise amplifier 402a and collected at a first ECG/Resp-dedicated data acquisition chip 403a; and the oxygen saturation signal is amplified by a second low-noise amplifier 402b and collected at a second oxygen saturation dedicated data acquisition chip 403b.

The data acquisition chips 403 convert the analog signals to digital signals and transmit the signals to a microcontroller 404 for digital filtering and processing for the output of an isolated ECG signal informing the infant's heart rate based on a QRS algorithm, an isolated respiratory signal informing the infant's respiratory rate, and an isolated oxygen saturation signal informing the infant's oxygen saturation rate. Each of the isolated signals are stored in a memory 405 and delivered to a data buffer 406 for transmission through an output 407 (e.g., a module connector) for presentation to a user through a user interface 408 (e.g., a display monitor). Preferably, the signals are output to the display 408 with a synchronized clock signal.

In the example shown in FIG. 7, biosignals are collected from the patient directly by the system 400; however, in other examples the biosignals may instead be collected by another device (e.g., a cardiopulmonary monitor, a pulse oximetry monitor, etc.) and then transmitted to the system 400 (e.g., through an input feeding to the amplifiers 402). Optionally, one or more automatic gain control units may be included for controlling signal amplification to enhance clarity of the collected biosignal waveforms.

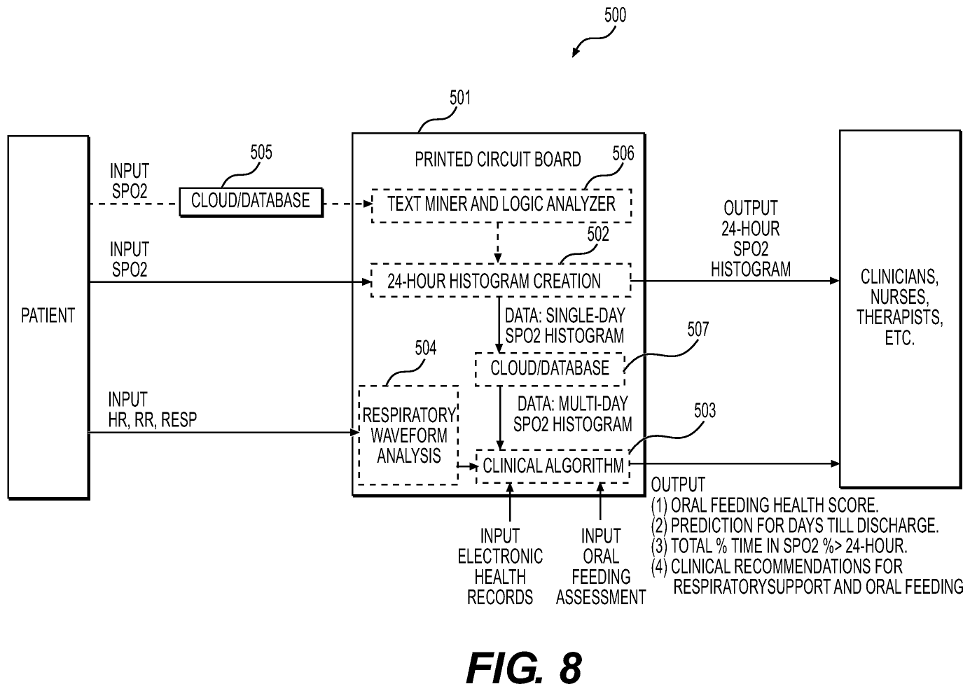

FIG. 8 shows an example of a system 500 according to the present invention, in the form of a system-on-chip built on a printed circuit board 501. The system 500 includes a histogram module 502 for generating oxygen saturation histograms that may be stored and consolidated over time, and a clinical algorithm module 503 for identifying correlations between oxygen saturation levels and respiratory waveforms to calculate a health score, generate a recommended treatment regimen, and/or predict a treatment outcome.

Preterm neonatal infants have been observed to have abnormal oxygen saturation levels (e.g., hyperoxia and hypoxia), which is associated with various morbidities including retinopathy of prematurity, bronchopulmonary dysplasia, neurodevelopmental impairment, and increased mortality. As such, oxygen monitoring is a standard of care in the NICU setting. However, such monitoring is traditionally performed simply by recording oxygen saturation percent of a patient at single isolated moments in time, for example by a bedside cardiopulmonary monitor, but the recorded data is not stored or made easily accessible to clinical caregivers.

The system 500 improves on these practices by creating and storing oxygen saturation histogram reports that visualize continuous oxygen saturation levels over a period of time, providing a more accurate assessment of a patient's oxygen stability. The system 500 may further cross-reference recorded oxygen saturation histogram reports with clinical outcomes, such as oral feeding performance and days to discharge, to identify correlations between of oxygen saturation patterns and an infant's readiness or progression toward discharge from the NICU.

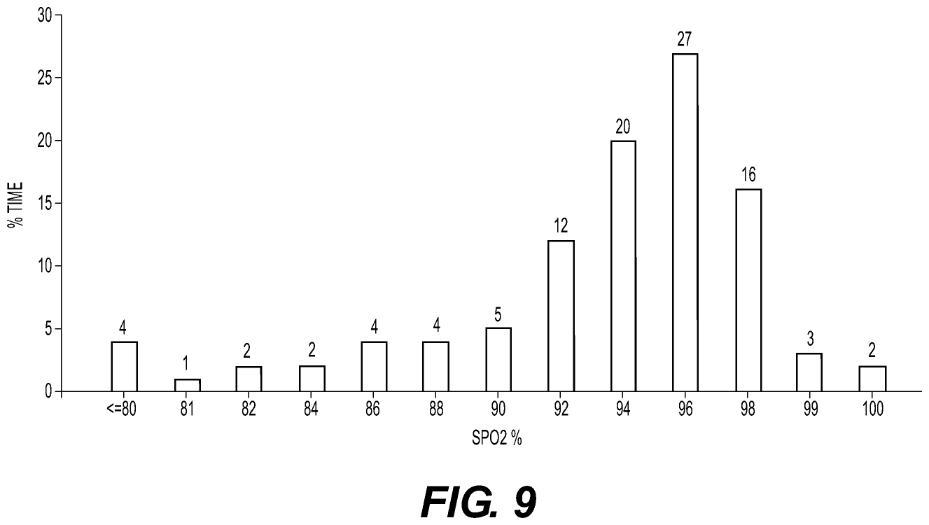

In the example shown in FIG. 8, a patient, such as a preterm infant, is monitored for capture of multiple biosignal inputs, including a first biosignal (Input 1) in the form of an oxygen saturation (SpO₂) signal, and a second biosignal (Input 2) that may be any one or a combination of a heart rate (HR), a respiration rate (RR), and a respiration pattern (RESP). The second biosignal (Input 2) is received at a waveform analysis module 504 where the input signal is processed in accord with the signal processing as discussed previously relative to step S204 in the method of FIG. 2. The oxygen saturation signal (Input 1) is received at the histogram module 502 that generates oxygen saturation histograms. Individual oxygen saturation histograms may cover a twenty-four-hour period of hourly blood oxygen saturation percentages graphed with percent of time, as shown in FIG. 9. The histogram module 502 may optionally generate histograms covering periods other than twenty-four hours, including shorter and longer periods.

The system 500 may incorporate multiple input means for reception of the oxygen saturation signal (Input 1), including a direct input of raw oxygen saturation data from oxygen saturation electrodes attached to the patient, and an indirect input of readings from an auxiliary source 505 storing oxygen saturation level readings previously taken from the patient. Examples of an auxiliary source 505 may include a local database (e.g., a bedside cardiopulmonary monitor) or a remote database accessible over a network communication (e.g., a cloud database).

When receiving an oxygen saturation signal (Input 1) from an auxiliary source 505, there is a possibility that the data relevant to oxygen saturation levels may have been stored at the auxiliary source 505 in a format (e.g., a portable document format, PDF) different from a format used by the histogram module 502 for generating oxygen saturation histograms. As such, oxygen saturation signals received from an auxiliary source 505 are first transmitted to a text-logic module 506 that is configured to extract relevant numerical data needed by the histogram module 502. The text-logic module 506 may employ document mining code, such as optical character recognition, and a logic algorithm to extract relevant numerical data and reformat the same into a format usable by the histogram module 502.

The histogram module 502 is programmed (e.g., via appropriate language such as Python, MATLAB, C++, etc.) to automatically and repeatedly generate oxygen saturation histograms over a predetermined period of time. Preferably, the predetermined period of time is a twenty-four-hour period, though the system 500 may optionally be adjusted to generate histograms over any other selected period of time, including shorter and longer periods. The histogram module 502 may generate histograms with a similar plotted format as that shown in FIG. 9, or in any other chosen format. Histograms generated by the histogram module 502 are stored in a memory 507, which may be in the form of a local storage space at the system 500 or a remote storage space apart from the system 500 (e.g., a cloud database). Optionally, the histogram module 502 may also directly output a generated histogram for presentation to clinical caregivers, for example, via a visual display component and/or a printing device.

In addition to storing newly generated oxygen saturation histograms at the memory 507, the histogram module 502 may also construct one or more consolidated histograms that combine one or more individual histograms to cover an extended period of time. For example, if the histogram module 502 is configured to generate individual histograms covering a twenty-four-hour period, then the histogram module 502 may also construct a composite histogram that covers a multi-day period that may correspond with a portion or the entire duration of an ongoing treatment period for the monitored patient.

The clinical algorithm module 503 receives one or more oxygen saturation histograms and/or consolidated histograms from the memory 507, along with processed waveforms from the waveform analysis module 504. The clinical algorithm module 503 may also receive any relevant patient data, such as that available from an EHR, as well as any additional data that might be available from other clinical monitoring efforts (e.g., oral feeding assessment). The clinical algorithm module 503 then calculates a health score, and generates and outputs a treatment protocol in accord with steps S206-S210 as discussed previously relative to the method of FIG. 2. As shown in the illustrated example, outputs from the clinical algorithm module 503 may include an oral feeding health score, prediction of days until discharge from the NICU, a histogram of oxygen saturation (with a total percent time of oxygen saturation in a pre-determined monitoring period), and clinical recommendations for respiratory support and oral feeding.

Optionally, the system 500 may be programmed to take one or more actions based on received biosignals and/or calculated health scores. In one such example, the analysis module 504 may be programmed to: verify that the patient is at the appropriate oxygen saturation percentage and duration; recommend respiratory support treatment modifications; and directly control the respiratory support to automate titration of oxygen in a closed system that provides personalized care. For example, when calculating a health score of a patient based at least in part on an oxygen saturation level, the clinical algorithm module 503 may compare a measured oxygen saturation level of the patient to a predetermined target range and take a predetermined action if the measured oxygen saturation level is outside a predetermined acceptable range.

The following Table I sets forth one example of predetermined conditions under which the system 500 may take action based on an oxygen saturation reading.

TABLE I
Oxygen Saturation (SpO₂)

| | Target | Lower | Upper |
|---|---|---|---|
| Classification | Range | Threshold | Threshold |
| BW ≤ 1500 g, and/or | 89-93 | 87 | 95 |
| PMA ≤ 32 weeks | | | |
| BW ≤ 1500 g, and/or | 93-98 | 90 | 99 |
| PMA = (33 to 36) weeks | | | |

BW = Birth Weight;
PMA = Postmenstrual Age

The system 500 may be configured to take an action when the oxygen saturation (SpO₂) is either below a lower threshold value or above an upper threshold value for a corresponding target range in which the monitored patient is classified. For example, the system 500 may act to trigger and alarm when the oxygen saturation is determined to be outside one of the threshold values, or may assume direct control of a respiratory support system to automatically titrate an oxygen supply to the patient. In some examples, the system may be programmed with two levels of threshold values at both the lower and upper limits, with a first threshold level being linked with only an alerting action (e.g., sounding an alarm), and the second threshold level being linked with a remedial action, such as an automated control of a patient support system (e.g., titration of oxygen supply).

By monitoring oxygen saturation levels to generate and consolidate oxygen saturation histograms, the system 500 is expected to further strengthen health score calculations and facilitate successful weaning of infants from ventilator support with decreased morbidities and mortalities.

Systems according to the present invention may serve as diagnostic aids to guide clinical decision making, and may provide diagnostic predictions and support for establishing clinical and therapeutic recommendations. In some examples, may be provided as diagnostic aids to guide clinical decision making. In such examples, the system may be employed as a clinical decision support tool powered by an AI-based computational algorithm (interpretive model). The computational algorithm may operate to synchronize infant breathing patterns with oral feeding performance, interpret the breathing patterns, and returns a metric that translates clinically into a rating on the progression of the preterm infant's feeding development which may then be used to guide clinical decision making for further treatment of the infant. The system may use a feature matrix to determine whether the infant demonstrates rigid or adaptable breathing patterns during oral feeding, and a clinician may then use that information when determining preferred treatment recommendations moving forward. Systems according to this example may be provided as a plug-in module, compatible with existing health monitoring systems used in the NICU, or as a stand-alone hand-portable device that is operable independently of any other health monitoring systems.

Systems according to the present invention may also use an interpretive model that is modified and tested through adaptive learning such that the clinical algorithm uses clinically guided machine learning studies to generate risk scores and provide guidance on patient care (e.g., a predictive model). In such examples, the system may create a static dataset that informs a predicted outcome and provides supervised learning (machine learning). The system may iteratively trend the dataset and improve the model by continually retraining the dataset. Systems according to this example may use the feature matrix to generate a diagnostic risk assessment score that informs a predicted feeding outcome for the infant, and which may be used by a clinician in providing clinical recommendations.

Systems according to the present invention may further use a machine learning model with a cloud-based data collection system for continually improving predictive accuracy based on new data (e.g., retraining the model) and artificial intelligence. Such continually adapting systems may provide guidance on treatment recommendations that have been observed to yield superior results based on an infant's specific parameters. For example, these systems may help to identify preferred medical interventions for recourse based on the infant's specific parameters, which may include changes to specific variables such as respiratory, oxygen and/or pharmacological support; or therapeutic interventions which may include modifications to nipple flow rate; oral feeding schedule; duration of feeding; volume of oral intake; feeding position; and/or feeding pacing.

It is expected that systems and methods according to the invention will enable clinicians to link oral feeding skills with non-invasive biomarkers so as to reduce complications, shorten hospitalizations, and improve the health, nutrition and overall outcomes for preterm infants by providing therapeutic regimens that are optimized to the needs of individual infants on a case-by-case basis.

The clinical application of the present inventions to monitoring respiratory waveforms using nonlinear methods will quantify the relationship between swallowing and breathing to help identify readiness for initiating and advancing oral feeding in preterm infants. For example, translating respiratory patterns into real-time information, further clinical insights will be available into the behavioral rigidity and/or disorganization of breathing patterns of a preterm infant during feed attempts, which may then be analyzed for assessing viability of current feeding regimes and determining if alternative action is needed. By assessing whether variability exists with the capacity to respond to unpredictable stimuli and stresses during oral feeding, variability monitoring may be helpful in assisting infants with oral feeding problems by allowing therapeutic interventions based on the process of coordination or variability of waveform pattern. Such interventions may contribute to improved clinical outcomes during oral feeding in preterm infants. Benefits for the infant may include, though are not limited to: improved cardiopulmonary response to enteral feeding; reduced fatigue with oral feeding; and better nutritional intake.

It will be understood of course that the invention is not limited to treatment of preterm infants or ailments effecting oral feeding, and that the inventions may apply to patients other than infants, and may apply to other physical and/or neurological disorders other than the mechanics of oral feeding, for improving consistency of care and individualized therapeutic interventions, as well as overall patient outcomes, generally.

Though the foregoing disclosure addresses certain examples, it will be understood that those examples are non-limiting. The present invention may be a system, a method, and/or a computer program product at any possible technical detail level of integration. The computer program product may include a computer readable storage medium (or media) having computer readable program instructions thereon for causing a processor to carry out aspects of the present invention. The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing.

A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other frecly propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers, and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to examples of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

Flowcharts and block diagrams in the figures illustrate examples of the architecture, functionality, and operation of possible implementations of systems, methods, and computer program products according to the present invention. Each block in a flowchart or block diagram may represent a module, segment, or portion of instructions, which comprises one or more executable instructions for implementing the specified logical function(s). In some alternative implementations, the functions noted in the blocks may occur in an order other than that shown in the figures. For example, two blocks shown in succession may, in fact, be executed substantially concurrently, or the blocks may sometimes be executed in the reverse order, depending upon the functionality involved. It will also be noted that each block of a block diagrams and/or flowchart illustration, and combinations of blocks in block diagrams and/or flowchart illustrations, can be implemented by special purpose hardware-based systems that perform the specified functions or acts or carry out combinations of special purpose hardware and computer instructions.

To the extent necessary to understand or complete the disclosure of the present invention, all publications, patents, and patent applications mentioned herein are expressly incorporated by reference herein to the same extent as though each were individually so incorporated. No license, express or implied, is granted to any patent incorporated herein.

Although the present invention is described with reference to particular embodiments, it will be understood to those skilled in the art that the foregoing disclosure addresses exemplary embodiments only; that the scope of the invention is not limited to the disclosed embodiments; and that the scope of the invention may encompass additional embodiments embracing various changes and modifications relative to the examples disclosed herein without departing from the scope of the invention as defined in the appended claims and equivalents thereto.

1. A system for guiding clinical treatments, comprising:
input circuitry adapted for receiving biosignals from at least one biosignal sensor that is adapted to transmit biosignals of a monitored patient;
at least one processor adapted for processing received biosignals to generate a treatment protocol for the monitored patient; and
output circuitry for outputting the treatment protocol;
wherein the input circuitry is adapted to receive biosignals as measured from a monitored patient in the form of a preterm infant during an oral feeding activity; and
wherein the treatment protocol comprises a recommended regimen for guiding clinical care of the monitored infant, the recommended regimen comprising one or more clinical interventions for a clinical caregiver to follow for promoting a predetermined outcome for improved oral feeding of the monitored infant.

2. The system according to claim 1, wherein:
the input circuitry is adapted to receive biosignals as waveforms;
the processor is adapted to process a received biosignal waveform to clean useful data from the biosignal waveform, calculate a health score from the cleaned biosignal waveform, and generate a treatment protocol based on the health score via a clinical algorithm that applies predefined rules.

3. The system according to claim 2, wherein:
the input circuitry is adapted to receive biosignal waveforms in the form of respiratory waveforms; and
the processor is adapted to generate a treatment protocol comprising a recommended regimen that comprises one or more clinical interventions in the form of therapeutic interventions and/or medical interventions.

4. The system according to claim 2, wherein:
the input circuitry is adapted to receive biosignal waveforms in the form of respiratory waveforms; and
the system further comprises one or more biosignal sensors connected to the input circuitry and adapted for non-invasive monitoring of the monitored infant, the one or more biosignal sensors being selected from: a body position sensor; a pulse oximeter; a blood pressure sensor; a temperature sensor; an electromyography (EMG) sensor; an electrocardiogram (ECG) sensor; an airflow sensor; and a galvanic skin response (GSR) sensor.

5. The system according to claim 3, wherein:
the processor is adapted to generate a treatment protocol through a series of steps that comprises:
matching the received biosignal with a corresponding treatment outcome;
interpreting the received biosignal as a rating of the patient's progress toward the corresponding treatment outcome;
cross-referencing the rating of the received biosignal with pre-established records for biosignals of the same type, the pre-stablished records comprising various ratings for the biosignal, treatment outcomes associated with the various ratings of the biosignal, and therapeutic interventions and/or medical interventions associated with the various ratings of the biosignal; and
outputting one or more therapeutic interventions and/or medical interventions associated with the interpreted rating of the received biosignal.

6. The system according to claim 5, wherein:
the processor is adapted to generate a treatment protocol in which:
the biosignal is matched with a treatment outcome in the form of preterm infant feeding outcomes;
the pre-stablished records comprise various ratings for respiratory waveforms measured during oral feeding activity, feeding associated with the various measured respiratory waveforms, and therapeutic interventions and/or medical interventions for achieving a targeted feeding outcome based on the various measured respiratory waveforms.

7. The system according to claim 6, wherein:
the processor is adapted to use data from an electronic health record of the monitored infant to generate a feature matrix that informs a rigidity or adaptability of the preterm monitored infant's breathing patterns during oral feeding.

8. The system according to claim 7, wherein:
the feature matrix is inclusive of one or more factors selected from: infant demographics, maternal factors, physiological factors, and measures of feeding performance.

9. The system according to claim 2, wherein:
the processor is adapted to calculate a health score (SampEn) based on the equation
$$SampleEn = {Φ}^{m} ( r ) = \frac{1}{N - m + 1} {\sum }_{i = 1}^{N - m + 1} log ( {C}_{i}^{m} ( r ) ) ,$$
in which N represents a length of a total time series of a dataset, m represents the length of a data segment, r represents a pre-established criterion for identifying similarity between data points, and Cᵢ^{m} represents a conditional probability.

10. The system according to claim 2, wherein:
the processor is adapted to take one or more actions based on received biosignals and/or calculated health scores.

11. The system according to claim 10, wherein:
the processor is adapted to take an action when a biosignal and/or health score is either below a lower threshold value or above an upper threshold value for a predetermined corresponding target range of the biosignal and/or health score.

12. The system according to claim 2, wherein:
the processor is adapted to clean useful data from a received biosignal waveform through a series of steps that comprises:
setting a defined waveform range for valid data, in which values within the defined waveform range are deemed valid data and values outside the defined waveform range are deemed potential artifact data;
identifying data points in a received biosignal waveform that are outside the defined waveform range, and designating the identified data points as potential artifact data points (PADP);
setting an earlier occurring PADP in the received biosignal waveform as a first data point DP₁, and setting a next successive occurring PADP after the earlier occurring PADP as a second data point DP₂;
determining if data defined by the data points DP₁ and DP₂ is valid data or artifact data by calculating a time (t) between the two data points DP₁ and DP₂, comparing the calculated time (t) to a predetermined time (T), and
if (t>T), designating the data points DP₁ and DP₂, and all data therebetween as valid data; and
if (t≤ T), designating the data points DP₁ and DP₂, and all data therebetween as artifact data.

13. The system according to claim 12, wherein:
the processor is adapted, following a determination as to the validity of a set of data points DP₁ and DP₂, to determine if there are any further data points that have been designated as PADP, and
if there is a further successive occurring PADP, change the data point that was previously set as the second data point DP₂ to now be set as the first data point DP₁ and set the further successive PADP as the second data point DP₂, and repeat the series of steps for cleansing useful data with the newly set data points DP₁ and DP₂; and
if there is not any further successive occurring PADP, terminate the cleansing of useful data from the received biosignal waveform.

14. The system according to claim 12, wherein:
the input circuitry is adapted to receive biosignal waveforms in the form of respiratory waveforms;
the processor is adapted for setting the defined waveform range to encompass respiratory-related movements of an infant, with values outside the defined waveform range encompassing non-respiratory movements of an infant; and
the predetermined time (T) is set based on expected durations of an infant's non-respiratory movements.

15. The system according to claim 1, wherein:
the input circuitry is adapted to receive biosignals in the form of one or more chosen from: respiratory rates and/or patterns; heart rates and/or patterns; brainwave patterns; muscle contraction patterns; eye movement patterns; partial pressure of oxygen (PaO₂); peripheral capillary oxygen saturation (SpO₂) patterns; arterial oxygen saturation (SaO₂) patterns; percent hemoglobin oxygen saturation patterns; oxygen saturation histogram patterns; blood pressure patterns; body temperature patterns; transcutaneous carbon dioxide tension (tcPCO₂) patterns; oxygen tensions (tcPO₂) patterns; and electrodermal activity patterns.

16. The system according to claim 1, wherein:
the treatment protocol comprises a recommended regimen for guiding clinical care of the monitored infant, the recommended regimen comprising one or more clinical interventions chosen from: respiratory support; oxygen support; pharmacological support; nipple flow rate; oral feeding schedule; duration of feeding; volume of oral intake; feeding position; and feeding pace.
